(19) United States
(12) Patent Application Publication
(10) Pub. No.: US 2026/0248990 A1
(43) Pub. Date: Aug. 27, 2026

(54) Stand Alone Cylindrical Blood Heat Exchanger With Spiral Volute

(71) Applicant: Breethe, Inc., Halethorpe, MD (US)

(72) Inventor: Charles Luddy, Danvers, MA (US)

(73) Assignee: Breethe, Inc., Halethorpe, MD (US)

(21) Appl. No.: 19/545,661

(22) Filed: Feb. 20, 2026

Related U.S. Application Data
(60) Provisional application No. 63/763,451, filed on Feb. 26, 2025.

(51) Int. Cl. A61M 1/16; A61M 60/113; A61M 60/38

(52) U.S. Cl. CPC A61M 1/1698; A61M 60/113; A61M 60/38; A61M 2205/366

(57) ABSTRACT

A standalone blood heat exchanger includes a first fluid path containing a heated/cooled liquid, a second fluid path containing blood, a network of hollow tubes arranged in an annular cylinder defining the first fluid path, a cylindrical body containing the annular cylinder having an interior wall, a defined space between the inner wall of the cylindrical body and an outer surface of the annular cylinder, and a central volume at the center of the annular cylinder, the second fluid path between the defined space and the central volume, passing through spaces between the hollow tubes. The defined space reduces stagnation of flow of the patient’s blood along the second fluid path. A geometric surface between the cylindrical body’s inner wall and its upper surface and lower surfaces reduces stagnation of blood flow within the defined space between the inner body wall and an outer surface of the annular cylinder.

### CROSS-REFERENCE TO RELATED APPLICATIONS

This application claims the benefit of the filing date of United States Provisional Patent Application No. 63/763,451 filed February 26, 2025 the disclosure of which is hereby incorporated herein by reference.

### BACKGROUND

The present invention relates to extracorporeal life support (ECLS), including both extracorporeal membrane oxygenation (ECMO) and cardiopulmonary bypass (CPB), and more particularly to a system for providing heating to blood in an ECLS system.

ECLS therapy operates by drawing deoxygenated blood from the body of a patient in a controlled manner to permit oxygenation of the blood and to remove carbon dioxide. The oxygenated blood is then cycled back into the patient. Generally, ECLS systems include a pump, an oxygenator, an oxygen source, and a control center to monitor and control the process. It may also include a blood warmer to bring the blood back up to the temperature of the body as it is circulated back into the body. Deoxygenated blood is drawn from the body via a cannula and is pumped through an oxygenator where oxygen is infused in the blood and carbon dioxide is removed from the blood. The oxygenated blood is pumped from the oxygenator and back into the body, with blood warming in most instances.

### SUMMARY

Aspects of embodiments described in this disclosure include a standalone blood heat exchanger having a first fluid path containing a heated or cooled liquid, a second fluid path containing blood from a patient, an annular cylinder comprising a network of hollow tubes oriented longitudinally along the length of the annular cylinder. The interior of the hollow tubes defines the first fluid path. A cylindrical body containing the annular cylinder and having an interior wall defining a space between the inner wall of the cylindrical body and an outer surface of the annular cylinder. A central volume runs longitudinally at the center of the annular cylinder, where the second fluid path includes the defined space between the inner wall of the cylindrical body and the outer surface of the annular cylinder and the central volume and the second flow path passing through spaces between the hollow tubes. The defined space between the inner wall of the cylindrical body and the outer surface of the annular cylinder is selected to reduce stagnation of flow of the patient’s blood while the patient’s blood travels along the second fluid path. The cylindrical body includes a circular upper surface abutting an end of the cylindrical inner wall of the cylindrical body and a circular lower surface abutting second end of the inner wall of the cylindrical body. A geometric surface is disposed between the inner wall of the cylindrical body and the upper surface and the lower surface, the geometric surface serving to reduce stagnation of the blood flow within the defined space between the inner wall of the cylindrical body and an outer surface of the annular cylinder. Blood enters the blood heat exchanger through a spiral volute disposed on an outer wall of the cylindrical body. The spiral volute has a slot extending through the outer wall of the cylindrical body where the slot allows the patient’s blood to enter the defined space between the inner wall of the cylindrical body and an outer surface of the annular cylinder. The slot in the spiral volute may have a decreasing width from the inlet end of the spiral volute to the opposing end of the spiral volute to evenly introduce the patient’s blood to the defined space between the inner wall of the cylindrical body and an outer surface of the annular cylinder. An inlet for receiving the heated or cooled liquid is located at the first end of the annular cylinder while an outlet for discharging the heated or cooled liquid at a opposite second end of the annular cylinder. The patient blood received at the inlet for receiving the patient’s blood is at a temperature of about 25 degrees Celsius to about 40 degrees Celsius. The patient blood discharge at the outlet for discharging the patient’s heated blood is at a temperature of about 25 degrees Celsius to about 40 degrees Celsius. According to an example of a method for heating blood in an extracorporeal life support (ECLS) system, the method includes positioning a standalone blood heat exchanger in line with a pump lung unit (PLU), directing patient blood through a first fluid path of the standalone blood heat exchange, directing a heated fluid through a second fluid path of the standalone blood heat exchanger where the first fluid path including a space defined between an inner wall of a cylindrical body of the standalone heat exchanger and an outer surface of an annular cylinder within the cylindrical body configured to reduce stagnation of flow of patient blood entering the first fluid path. Additionally, the method may further define a geometric shape between the inner wall of the cylindrical body and an upper surface of the cylindrical body and a lower surface of the cylindrical body. The geometric shape is selected from one of a fillet and a chamfer.

### BRIEF DESCRIPTION OF THE DRAWINGS

FIG. 1 is a cross-section view of a standalone blood heat exchanger according to aspects of embodiments described in this disclosure.

FIG. 2 is a cross-section view of fluid flow in a blood heat exchanger according to aspects of embodiments described in this disclosure.

FIG. 3 is an isometric view of a standalone blood heat exchanger with a blood oxygenator according to aspects of embodiments described in this disclosure.

FIG. 4 is a process flow diagram for heating blood in a standalone blood heat exchanger according to aspects of embodiments of this disclosure.

### DETAILED DESCRIPTION

Patients awaiting or recovering from a heart or lung transplant and patients having conditions putting them at risk for heart or lung failure may be candidates for extracorporeal life support (ECLS). ECLS therapy is designed to address an excess of carbon dioxide and/or lack of oxygen in the blood where the lungs are not healthy and/or the heart cannot pump enough blood around the body. ECLS is thus an extracorporeal technique which replaces these respiratory and cardiac functions in various situations ranging from support needed while treating the underlying causes of cardiac arrest to late-stage treatment for heart or lung failure. It is a therapeutic treatment to provide temporary help when needed. It does not address the underlying condition.

As alluded to herein, a blood heat exchanger is used to actively control blood temperature to meet the clinical core temperature needs of a patient during ECLS therapy. In conventional solutions, the blood heat exchanger is typically integrated with a pump lung unit (PLU), which oxygenates the blood. Accordingly, the blood heat exchanger cannot be changed or removed without disabling the PLU. It is imperative that blood passing through the PLU and heat exchanger maintains sufficient velocity to prevent stagnation and clotting, which can cause thrombus.

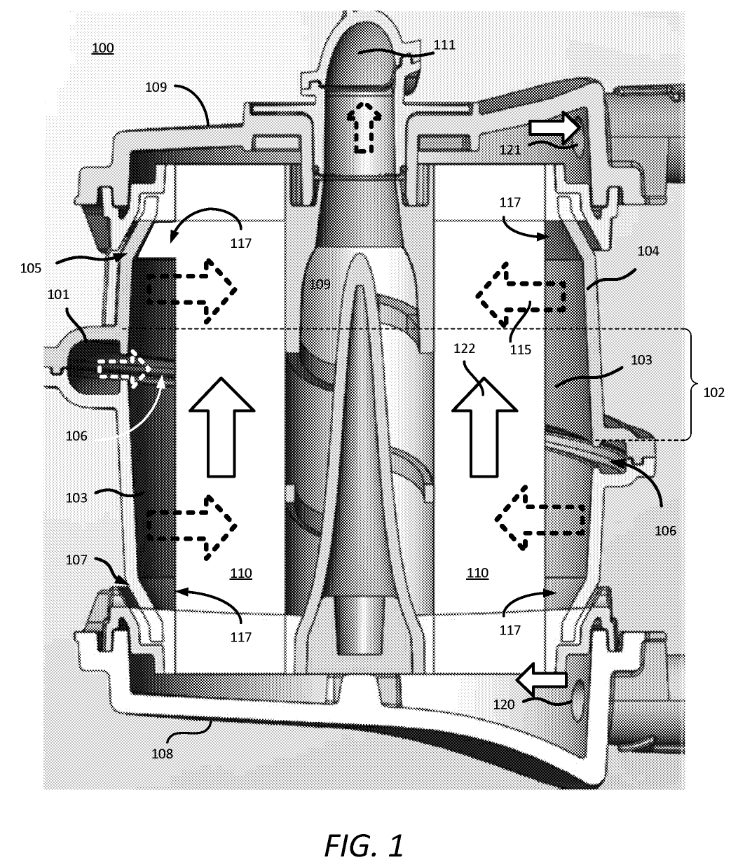

FIG. 1 is a cross-section view of a standalone blood heat exchanger 100 according to aspects of this disclosure. The standalone blood heat exchanger 100 defines two independent fluid paths. In a first fluid path, a heated or cooled liquid (e.g., water) flows. In the second fluid path, the patient’s blood flows and crosses in proximity to the first fluid path. The heated or cooled liquid in the first fluid path releases some of its heat to the patient’s blood in the second fluid path.

In the first fluid path, a heated or cooled liquid is introduced to the blood heat exchanger 100 at inlet 120. Upon entering the blood heat exchanger 100, the heated or cooled liquid contacts a first end of an annular cylinder 110. The annular cylinder 110 is made up of a network of hollow tubes that extend longitudinally from one end of the blood heat exchanger 100 to the opposite end. The heated or cooled liquid passes through the hollow tubes in direction 122 and exits the blood heat exchanger 100 at the top of FIG. 1 via outlet 121. The annular cylinder 110 is capped on its ends and the tube structure is potted into the end caps. The first fluid path includes inlet 120, the space in the blood heat exchanger 100 housing adjacent to bottom surface 108, through annular cylinder 110 to an upper volume of the blood heat exchanger housing defined by upper surface 109 and finally through outlet 121.

The hollow tubes that form the annular cylinder 110 terminate at each end of the annular cylinder 110 where the tube ends are potted to form opposing ends of the annular cylinder 110. The heated fluid is introduced at the ends of the hollow tubes at a first end of the annular cylinder 110, pass through the network of hollow tubes and exit the annular cylinder 110 at the opposing second end of the annular cylinder 110.

The second fluid path enters the blood heat exchanger 100 via a side wall of the blood heat exchanger housing through spiral volute 101. Spiral volute is disposed around the circumference of the cylindrical body 104 and follows an inclined plane along the length of the cylindrical body 104 defining a distance 102 between the inlet of the spiral volute 101 and its termination at the cylindrical body 104 wall. The spiral volute 101 includes a slot 106 defined through the wall of the body 104 of the blood heat exchanger 100. The slot 106 in spiral volute 101 varies in width along the length of the spiral volute 101 to provide even flow of blood into the internal volume 103 of the housing. Blood enters the blood heat exchanger 100 at inlet to spiral volute 101 and flows into and fills volume 103 of the cylindrical body 104 of the blood heat exchanger 100. Blood in the volume 103 between the outer wall of the body 104 and the outer surface of annular cylinder 110 passes across 115 the hollow tubes defining the annular cylinder 110. As the blood passes across 115 the annular cylinder 110, heat from the heated fluid passing through the annular cylinder 110 in direction 122 is conducted through the walls of the hollow tube to the blood passing across 115 the annular cylinder 110. The blood receives the heat from the heated or cooled liquid producing heated blood entering central volume 109 at the center of annular cylinder 110. The heated blood exits the blood heat exchanger 100 at outlet 111. Outlet 111 transports the heated blood to the next stage of the ECLS process. In some cases, this may include returning heated oxygenated blood back to the patient.

The edge 105 defined between the inside wall of the cylindrical body 104 of the blood heat exchanger 100 and the top surface 109 can include a geometric shape that discourages stagnation of secondary blood flow in volume 103. Likewise, the edge 107 defined between the inside wall of the cylindrical body 104 of the blood heat exchanger 100 and the lower surface 108 a geometric shape serving the same purpose. The geometric shape may be selected to minimize blood flow stagnation. According to some embodiments, the distance between the inside wall of the heat exchanger 104 and the annular cylinder 110 may be from about 0.5mm to about 15mm. For example, the distance between the inside wall of the heat exchanger 104 and the annular cylinder 110 may be from about 2mm to about 8mm. By way of example, the edge 105, 107 may be configured as a concave fillet disposed between the inner wall of the blood heat exchanger body 104 and the housing top surface 109 and lower surface 108. In another example, the edge 105, 107 may be configured as a chamfer between the inner wall of the blood heat exchanger body 104 and the housing top surface 109 and lower surface 108. In each case, the sharp edges at the intersection of adjoining surfaces are softened, encouraging fluid flow and reducing the risk of stagnation.

According to one embodiment, the heated or cooled fluid may enter the blood heat exchanger 100 at about 40º Celsius. Blood may enter the blood heat exchanger 100 at about 25º to about 40º Celsius passes across the heated fluid where heat is transferred to the blood. The blood may leave the blood heat exchanger 100 at a temperature of about 33º to about 40º Celsius. According to some embodiments, the standalone blood exchanger 100 may be a single use device, intended for a single patient, after which the device may be discarded.

As shown in FIG. 1, the heat exchanger 100 may be setup having a blood circuit flow of 5L/min and a water circuit flow of 10L/min. In some embodiments, the heat exchanger 100 may have a blood circuit flow of about 0 L/min to about 10 L/min. In other embodiments, the heat exchanger 100 may have a water circuit flow of about 0 L/min to about 30 L/min. Blood may enter the heat exchanger 100 at 30º C while the heated or cooled water may enter the water circuit at 40º C. In some embodiments, the heat exchanger may require a priming volume of about 163 cc, while the residual blood in the heat exchanger may be limited to about 32.8 cc. As will be appreciated, the heat exchanger may be any suitable size and/or require any suitable priming volume. Stagnation regions may be reduced and/or eliminated when sufficient space exists between the inner wall of the housing body 104 and the outer wall of annular cylinder 110 and where secondary draft angles 117 are softened at the ends of the annular cylindrical body 104 where the sides meet the upper surface 109 and lower surface 108. In some embodiments, the secondary draft angles 117 may be between about 0 degrees and about 45 degrees. More specifically, the secondary draft angles 117 may be between about 2 degrees and about 30 degrees. In other embodiments, the geometry of the ends of the annular cylindrical body 104 where the sides meet the upper surface 109 and the lower surface 108 may be a chamfer. In some embodiments, the geometry of the ends of annular cylindrical body 104 may be a fillet. As will be appreciated, the ends of annular cylindrical body 104 may be of any suitable geometry.

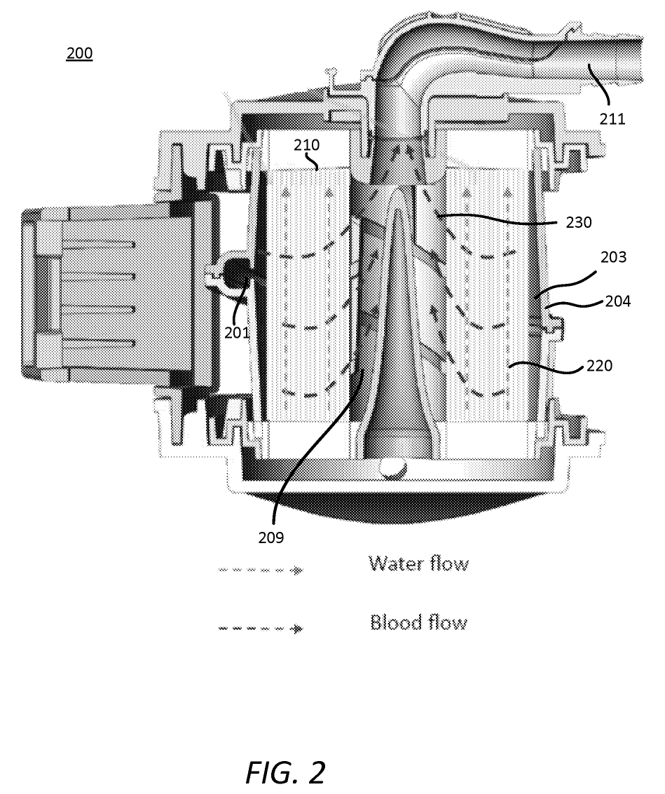

FIG. 2 is a cross-section view of a blood heat exchanger 200 illustrating the general flow of fluids through the blood heat exchanger 200. A first flow path 220 is defined through a system of hollow tubes that receive a heated or cooled liquid that passes longitudinally through an annular cylinder defined by the system of hollow tubes. A second flow path 230 enters the blood heat exchanger 200 through a body 204 defining a volume 203 around the annular cylinder 210 via spiral volute 201. The second flow path 230 crosses the annular cylinder 210, the second flow path 230 passing between the hollow tubes making up the annular cylinder 210, entering a central volume 209 and exiting the blood heat exchanger 200 through outlet 211.

Water or other heated fluid passes longitudinally along annular cylinder 210 through a network of hollow tubes. The water or other heated fluid enters the first flow path 220 at a temperature greater than that of the blood entering the heat exchanger 200 through spiral volute 201. As the blood follows second flow path 230 passing between the hollow tubes of annular cylinder 210 containing the water or heated fluid, the heat in the water or heated fluid is transferred across the surfaces of the hollow tubes and absorbed by the passing blood in second flow path 230. The warmed blood exits the heat exchanger 200 via outlet 211 at a temperature that is compatible with the core temperature of the patient.

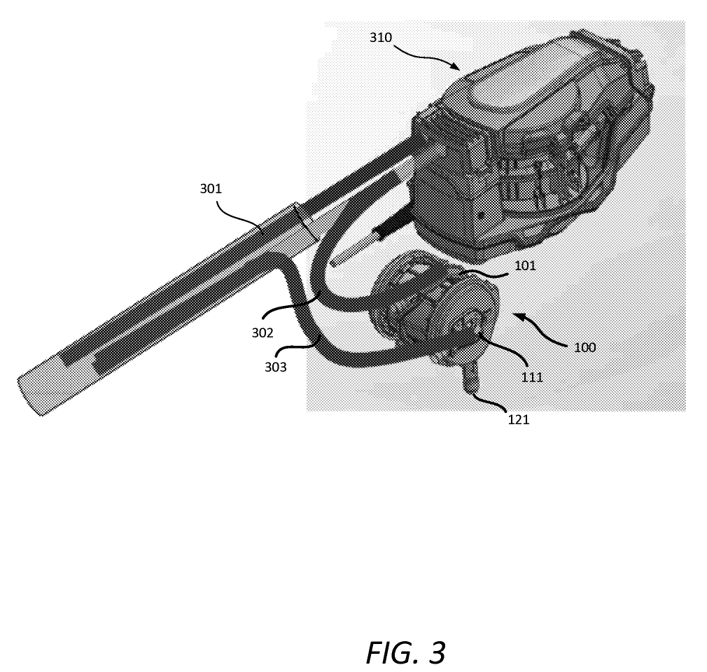

FIG. 3 is an isometric view of a blood oxygenation system according to aspects of embodiment of this disclosure. Blood is directed from a patient along input path 301 and enters blood oxygenator 310. Blood oxygenator 310 exposes the patient blood to an oxygen rich gas source and transfers oxygen into the blood. The oxygenated blood leaves the oxygenator 310 via tubing 302 that enters standalone blood heat exchanger 100 via spiral volute 101. A heated fluid is introduced to blood heat exchanger 100. The blood enters the blood heat exchanger 100 at spiral volute 101 and passes across the flow of the heated or cooled liquid to transfer heat from the heated fluid to the blood. The heated fluid exits the blood heat exchanger 100 through outlet 121. The heated blood leaves the blood heat exchanger 100 through a centrally located outlet 111 and is discharged through tubing 303 and returns to the patient. The returned blood is oxygenated and heated to meet the physiological needs of the patient. As will be appreciated, the blood heat exchanger 100 may be located in any suitable location within the blood oxygenation system.

Heat exchanger 100 is configured as a standalone device, independent of the blood oxygenator 310. The heat exchanger 100 and blood oxygenator 310 can be mounted relative to each other on a vertical support such as a portable intravenous (IV) pole. In one embodiment, the blood oxygenator 310 is mounted above the standalone blood heat exchanger 100. The blood heat exchanger 100 is connected to the blood oxygenator 310 by the blood flow circuit of the heat exchanger 100 via tubing 302. The two components may be positioned relative to one another to maintain portability of the system, such as on an ambulatory IV pole. This allows a patient to move about while receiving ECMO therapy having the ability to move the entire functional ECLS system without having to remain bedridden, supporting the overall health and recovery of the patient.

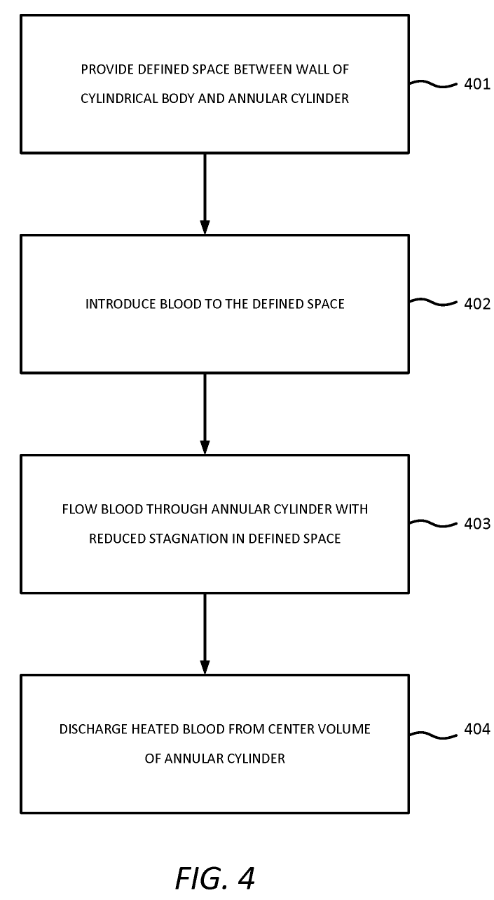

Referring now to FIG. 4, a process flow diagram is provided that illustrates a method of operating a standalone blood heat exchanger according to aspects of embodiments of this disclosure. The blood heat exchanger includes a body defining a main volume. Within the main volume, a first fluid path is defined by a system of hollow tubes arranged together to form an annular cylinder. A heated or cooled liquid is introduced at one end of the annular cylinder and passes through the hollow tubes longitudinally along the annular cylinder. Blood is introduced to the main volume and crosses the hollow tubes containing the heated or cooled liquid. Heat is transferred from the heated or cooled liquid to the blood. A volume is defined between the inner wall of a cylindrical body defining the main volume and the outer surface of the annular cylinder 401.

The blood is introduced to the blood heat exchanger in the defined space 402. The blood enters the blood heat exchanger via a spiral volute that is disposed around the wall of the cylindrical body of the blood heat exchanger. The spiral volute defines a slot through the wall of the cylindrical body. The slot has a varying width along the length of the spiral volute to equalize the flow of blood into the space inside the cylindrical body. The defined volume around the annular cylinder to the inner wall of the cylindrical body is provided to minimize or eliminate stagnant areas of blood flow within the cylindrical body.

The comparative cross-sectional diameter of the cylindrical body relative to the annular cylinder may be selected to reduce or minimize stagnation regions in the defined space between the cylindrical body and the annular cylinder 403. Providing sufficient volume between the annular cylinder and the body wall reduces the occurrence of stagnation regions within the cylindrical body of the blood heat exchanger.

The blood is passed across the annular cylinder and enters a central volume of the annular cylinder. The annular cylinder contains heated fluid at a temperature above that of the blood. As the blood passes through the network of hollow tubes in the annular cylinder, the heat from the heated fluid is transferred to the blood. The heated blood is then discharged from the central volume 404 and exits the blood heat exchanger.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A standalone blood heat exchanger comprising:
a first fluid path containing a heated liquid;
a second fluid path containing blood from a patient;
an annular cylinder comprising a network of hollow tubes oriented longitudinally along the length of the annular cylinder, the interior of the hollow tubes defining the first fluid path;
a cylindrical body containing the annular cylinder, the cylindrical body having an interior wall and having a defined space between the inner wall of the cylindrical body and an outer surface of the annular cylinder; and
a central volume running longitudinally at the center of the annular cylinder, the second fluid path being disposed between the defined space between the inner wall of the cylindrical body and the outer surface of the annular cylinder and the central volume, passing through spaces between the hollow tubes, wherein the defined space between the inner wall of the cylindrical body and the outer surface of the annular cylinder is selected to reduce stagnation of flow of the patient’s blood while the patient’s blood travels along the second fluid path.

2. The standalone blood heat exchanger of claim 1, further comprising:
the cylindrical body having a circular upper surface abutting an end of the cylindrical inner wall of the cylindrical body; and
the cylindrical body having a circular lower surface abutting second end of the inner wall of the cylindrical body.

3. The standalone blood heat exchanger of claim 2, further comprising a geometric surface between the inner wall of the cylindrical body and the circular upper surface and the circular lower surface, the geometric surface serving to reduce stagnation of the blood flow within the defined space between the inner wall of the cylindrical body and an outer surface of the annular cylinder.

4. The standalone blood heat exchanger of claim 3, the geometric space defining a fillet between the inner wall of the cylindrical body and the upper and lower surfaces.

5. The standalone blood heat exchanger of claim 4, the geometric space defining the fillet between the inner wall of the cylindrical body and the upper and lower surfaces is a concave fillet.

6. The standalone blood heat exchanger of claim 3, the geometric space defining a chamfer between the inner wall of the cylindrical body and the upper and lower surfaces.

7. The standalone blood heat exchanger of claim 2 further comprising; a spiral volute disposed on an outer wall of the cylindrical body, the spiral volute having a slot extending through the outer wall of the cylindrical body the slot allowing the patient’s blood to enter the defined space between the inner wall of the cylindrical body and an outer surface of the annular cylinder.

8. The standalone blood exchanger of claim 7, the slot having a decreasing width from a first inlet end of the spiral volute to a second end of the spiral volute to evenly introduce the patient’s blood to the defined space between the inner wall of the cylindrical body and an outer surface of the annular cylinder.

9. The standalone blood heat exchanger of claim 7, further comprising an inlet for receiving the patient’s blood at a first end of the spiral volute at the outer wall of the cylindrical body.

10. The standalone blood heat exchanger of claim 9, further comprising an outlet for discharging heated patient’s blood in fluid communication with the central volume at the center of the annular cylinder.

11. The standalone blood heat exchanger of claim 10, the patient blood received at the inlet for receiving the patient’s blood is at a temperature of about 25 degrees Celsius to about 40 degrees Celsius.

12. The standalone blood heat exchanger of claim 10, the patient blood discharge at the outlet for discharging the patient’s heated blood is at a temperature of about 25 degrees Celsius to about 40 degrees Celsius.

13. The standalone blood heat exchanger of claim 12, wherein the patient blood discharge at the outlet for discharging the patient’s heated blood is at a temperature is compatible with a core temperature of the patient.

14. The standalone blood heat exchanger of claim 1, wherein the standalone blood heat exchanger is disposable and provided for a single use.

15. The standalone blood heat exchanger of claim 1, the annular cylinder having a first end and a second opposite end, the ends of the hollow tubes being potted into the first end and second end of the annular cylinder.

16. The standalone blood heat exchanger of claim 15, further comprising:
an inlet for receiving the heated liquid at the first end of the annular cylinder; and an outlet for discharging the heated liquid at the second end of the annular cylinder.

17. The standalone blood heat exchanger of claim 14, wherein the heated fluid at the inlet is received at about 20 degrees Celsius to about 40 degrees Celsius.

18. A method for heating blood in an extracorporeal life support (ECLS) system, comprising:
positioning a standalone blood heat exchanger in line with a pump and an oxygenator;
directing patient blood through a first fluid path of the standalone blood heat exchanger;
directing a heated fluid through a second fluid path of the standalone blood heat exchanger; and
the first fluid path including a space defined between an inner wall of a cylindrical body of the standalone heat exchanger and an outer surface of an annular cylinder within the cylindrical body configured to reduce stagnation of flow of patient blood entering the first fluid path.

19. The method of claim 18, further comprising:
defining a geometric shape between the inner wall of the cylindrical body and an upper surface of the cylindrical body and a lower surface of the cylindrical body.

20. The method of claim 18, wherein the geometric shape is selected from one of a fillet and a chamfer.
